(19) United States
(12) Patent Application Publication
(10) Pub. No.: US 2021/0177560 A1
(43) Pub. Date: Jun. 17, 2021

(54) ELECTRIC TOOTHBRUSH

(71) Applicant: Chul Kyun JUNG, Bucheon-si (KR)

(72) Inventor: Chul Kyun JUNG, Bucheon-si (KR)

(21) Appl. No.: 17/106,920

(22) Filed: Nov. 30, 2020

(30) Foreign Application Priority Data
Dec. 16, 2019 (KR) 10-2019-0167433

(51) Int. Cl. A61C 17/26

(52) U.S. Cl. CPC A61C 17/26

(57) ABSTRACT

An electric toothbrush includes a body part provided with an electric toothbrush head, and a cover part detachably coupled to the electric toothbrush head to sterilize the electric toothbrush head, and the body part is provided with contact type power output terminals to supply a power to the cover part. By including the cover part for sterilization configured to receive a power from a body part of the electric toothbrush with being coupled to the body part of the electric toothbrush, the electric toothbrush allows a user to perform sterilization treatment on the electric toothbrush by coupling the cover part to the body part after use of the electric toothbrush without a need to purchase a separate device for sterilization and carry the same, as well as allowing the user to easily perform hygiene management of the electric.

### RELATED APPLICATIONS

This application claims priority to Korean Patent Application No. 10-2019-0167433, filed on Dec. 16, 2019 in the Korean Intellectual Property Office, the entire disclosure of which is incorporated herein by reference.

### BACKGROUND OF THE INVENTION

### 1. Field of the Invention

The present invention relates to an electric toothbrush, and more particularly, to an electric toothbrush which includes a cover part for sterilization configured to receive a power from a body part of the electric toothbrush with being coupled to the body part of the electric toothbrush, thereby allowing a user to perform sterilization treatment on the electric toothbrush by coupling the cover part to the body part after use of the electric toothbrush without a need to purchase a separate device for sterilization and carry the same, as well as allowing the user to easily perform hygiene management of the electric toothbrush through the cover part for sterilization that is coupled to the body part of the electric toothbrush for storage after use of the electric toothbrush, such that portability of the electric toothbrush can be greatly improved.

### 2. Description of the Related Art

Recently, sonic electric toothbrushes are widely used for dental care and healthy gums. However, due to a failure of hygiene management of the electric toothbrush, there is a problem that bacteria propagate in a toothbrush head, which may adversely affect dental health.

Meanwhile, most electric toothbrushes are currently installed and used in homes and offices, and users are purchasing and using a separate device for sterilization which receives an external power for sterilization treatment of the toothbrush head.

Most of such conventional devices for sterilization of an electric toothbrush not only use an external power, but also have poor portability due to a large volume. Accordingly, there is a technical limitation in that it is difficult to carry along with the electric toothbrush when a user has a business trip or travels.

Such a technical limitation is also a reason why users mainly keep and use the electric toothbrush only in their homes or offices, and do not carry it while on a business trip or traveling.

### SUMMARY OF THE INVENTION

In consideration of the above-mentioned circumstances, it is an object of the present invention to provide an electric toothbrush which includes a cover part for sterilization configured to receive a power from a body part of the electric toothbrush with being coupled to the body part of the electric toothbrush, thereby allowing a user to perform sterilization treatment on the electric toothbrush by coupling the cover part to the body part after use of the electric toothbrush without a need to purchase a separate device for sterilization and carry the same, as well as allowing the user to easily perform hygiene management of the electric toothbrush through the cover part for sterilization that is coupled to the body part of the electric toothbrush for storage after use of the electric toothbrush, such that portability of the electric toothbrush can be greatly improved.

To achieve the above object, according to an aspect of the present invention, there is provided an electric toothbrush including: a body part which includes an electric toothbrush head; and a cover part detachably coupled to the electric toothbrush head to sterilize the electric toothbrush head.

Preferably, the body part includes contact type power output terminals configured to supply a power to the cover part.

In addition, the cover part may include contact type power input terminals which come into contact with the contact type power output terminal provided in the body part with being coupled to the electric toothbrush head.

Further, the body part may include a control module configured to control power supply to the power input terminals through the contact power output terminals.

Further, the cover part may include a sterilization lamp provided therein to emit light by the power supplied through the power input terminals.

Further, the cover part may include a sterilization indicator provided therein to emit light by the power supplied through the power input terminals.

Further, the control module may stop power supply through the power output terminals after a predetermined sterilization treatment time has elapsed from a time when the power output terminals and the power input terminals are in contact with each other.

Furthermore, the body part may further include an input module configured to input setting information of the sterilization treatment time.

Furthermore, the cover part may include slits formed therein to check a light emission state of the sterilization lamp.

According to the present invention, the electric toothbrush includes the cover part for sterilization configured to receive a power from the body part of the electric toothbrush with being coupled to the body part of the electric toothbrush, thereby allowing a user to perform sterilization treatment on the electric toothbrush by coupling the cover part to the body part after use of the electric toothbrush without a need to purchase a separate device for sterilization and carry the same.

In addition, according to the present invention, the user may easily perform hygiene management of the electric toothbrush through the cover part for sterilization that is coupled to the body part of the electric toothbrush for storage after use of the electric toothbrush, such that portability of the electric toothbrush can be greatly improved.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view illustrating a structure of a body part of an electric toothbrush according to an embodiment of the present invention;

FIG. 2 is a view illustrating a state in which a cover part is coupled to the body part for storage after use of the electric toothbrush shown in FIG. 1;

FIG. 3 is a view illustrating a structure of an inner surface of the cover part of the electric toothbrush according to an embodiment of the present invention;

FIG. 4 is a functional block diagram illustrating the structure of the cover part of the electric toothbrush according to an embodiment of the present invention; and

FIG. 5 is a functional block diagram illustrating the structure of the body part of the electric toothbrush according to an embodiment of the present invention.

### DETAILED DESCRIPTION OF THE INVENTION

Hereinafter, the present invention will be described with reference to the accompanying drawings in detail. Referring to the drawings, wherein like reference characters designate like or corresponding parts throughout the several views. In the embodiments of the present invention, the publicly known functions and configurations that are judged to be able to make the purport of the present invention unnecessarily obscure will not be described.

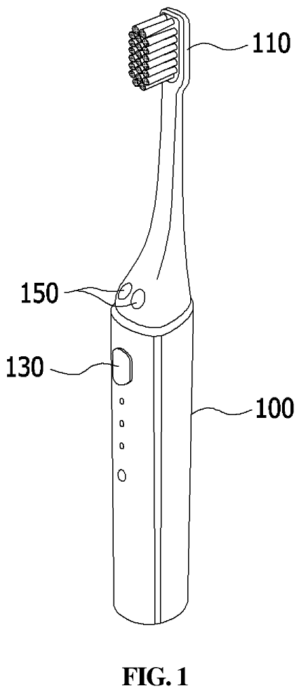

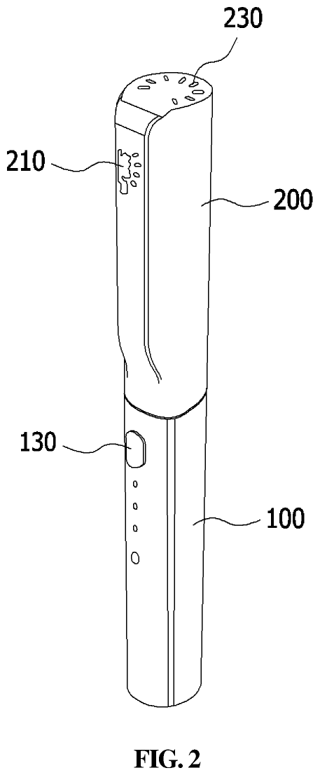

FIG. 1 is a view illustrating a structure of a body part 100 of an electric toothbrush according to an embodiment of the present invention, and FIG. 2 is a view illustrating a state in which a cover part 200 is coupled to a body part 100 for storage after use of the electric toothbrush shown in FIG. 1.

Referring to FIGS. 1 and 2, the electric toothbrush according to an embodiment of the present invention includes the body part 100 and the cover part 200, and the body part 100 of the electric toothbrush includes an electric toothbrush head 110, an input module 130, and power output terminals 150.

The electric toothbrush head 110 is provided at an upper portion of the cylindrical body part 100 grasped by a user with a hand, and is driven by a power supplied from a rechargeable battery module provided in the body part 100.

The input module 130 is provided in a central portion of a side of the body part 100. The user controls on/off operations of a sonic electric module provided in the electric toothbrush head 110 through a button provided in the input module 130.

Meanwhile, the rechargeable battery module provided in the body part 100 not only supplies a power for operation of the sonic electric module which drives the electric toothbrush head 110, but also, as shown in FIG. 2, supplies a power for operation of a sterilization lamp 270 provided in the cover part 200 for sterilizing the electric toothbrush head 110, which is detachably coupled to the electric toothbrush head 110.

Specifically, the power supplied from the rechargeable battery module provided in the body part 100 is supplied to the sterilization lamp 270 provided in the cover part 200 through the contact type power output terminals 150 provided on an upper side of the body part 100.

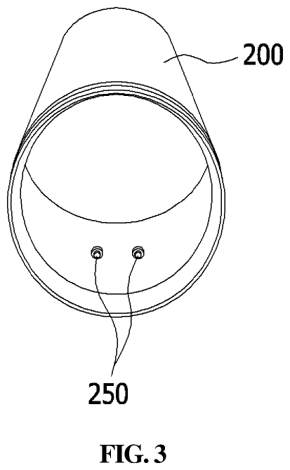

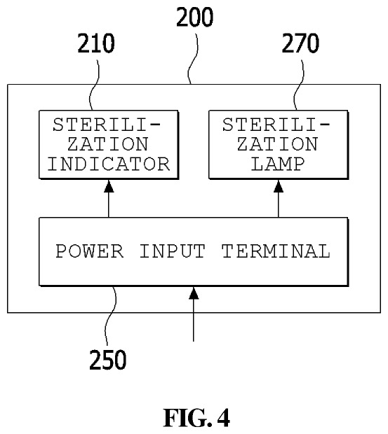

FIG. 3 is a view illustrating a structure of an inner surface of the cover part 200 of the electric toothbrush according to an embodiment of the present invention, and FIG. 4 is a functional block diagram illustrating the structure of the cover part 200 of the electric toothbrush according to an embodiment of the present invention.

Referring to FIGS. 3 and 4, the cover part 200 of the electric toothbrush according to an embodiment of the present invention includes a sterilization indicator 210, open slits 230, the sterilization lamp 270, and the power input terminals 250.

As shown in FIG. 3, the cover part 200 of the electric toothbrush is provided with the contact type power input terminals 250 at a lower end of an inner surface thereof. When the cover part 200 is coupled to the body part 100 by the user as shown in FIG. 2, a pair of contact type power output terminals 150 provided at an upper end of an outer surface of the body part 100 and a pair of contact type power input terminals 250 provided at the lower end of the inner surface of the cover part 200 come into contact with each other, such that the cover part 200 receives a power for operation from the body part 100.

Meanwhile, the sterilization lamp 270 such as an LED UV lamp which sterilizes the electric toothbrush head 110 is installed on the inner surface of the cover part 200, and the sterilization indicator 210, which is a lamp for indicating whether sterilization treatment is performed on the electric toothbrush head 110, is installed on the outer surface of the cover part 200.

Thereby, when the cover part 200 is coupled to the body part 100 by the user who has fmished using the electric toothbrush, the power input terminals 250 of the cover part 200 which receive a power from the power output terminals 150 of the body part 100 supply the power for operation supplied from the body part 100 to the sterilization lamp 270 and the sterilization indicator 210, respectively.

As described above, as the power is supplied from the power input terminals 250, the sterilization lamp 270 emits light, and thereby sterilizing the electric toothbrush head 110. At the same time, the sterilization indicator 210 also comes on, thereby indicating to an outside that the sterilization treatment is being performed on the electric toothbrush head 110.

Meanwhile, as shown in FIG. 2, the cover part 200 has a plurality of open slits 230 formed therein. Thereby, a part of the light irradiated according to light emission of the sterilization lamp 270 is exposed to the outside of the cover part 200 through the open slits 230.

As described above, in the present invention, the light emitted from the sterilization lamp 270 is exposed to the outside through the open slits 230 formed in an upper surface of the cover part 200. Therefore, even when the sterilization indicator 210 malfunctions, the user may not only accurately check an operation state of the sterilization lamp 270, but also perform interior functions such as indoor lighting through the electric toothbrush being sterilized.

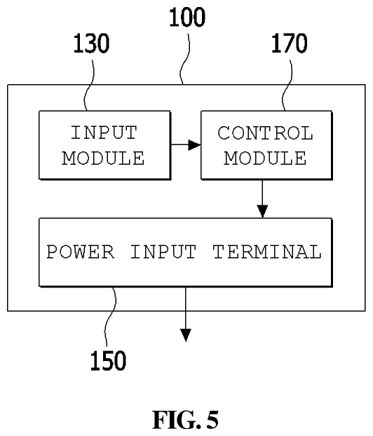

FIG. 5 is a functional block diagram illustrating the structure of the body part 100 of the electric toothbrush according to an embodiment of the present invention. Referring to FIG. 5, the body part 100 of the electric toothbrush according to an embodiment of the present invention may further include a control module 170, in addition to the power output terminals 150 and the input module 130 shown in FIG. 1.

The control module 170 controls power supply of the internal battery module to the sound wave electric module based on operation information on the electric toothbrush of the user input through the input module 130.

In addition, as shown in FIG. 2, when coupling the cover part 200 to the body part 100 by the user, and the power output terminals 150 provided in the body part 100 come into contact with the power input terminals 250 provided in the cover part 200 in a contact coupling manner, the control module 170 supplies the power of the internal battery module to the power input terminals 250 through the power output terminals 150.

Meanwhile, when a predetermined time (for example, 5 minutes) has elapsed from a time when the power supply from the body part 100 to the cover part 200 is started by coupling the cover part 200 to the body part 100 as described above, the control module 170 stops the power supply through the power output terminals 150. Accordingly, after the electric toothbrush head 110 is subjected to the sterilization treatment for a sufficient time, it is possible to prevent the power from continuously being supplied to the cover part 200.

As described above, for embodying the present invention, the control module 170 may have a timer function for a sterilization duration, which is a time to continue supplying power to the cover part 200. Herein, it is preferable that the user directly sets the sterilization duration through the button provided in the input module 130.

In addition, for embodying the present invention, the body part 100 may be provided with a communication module that supports wireless communication such as Bluetooth with a user terminal such as a smart phone, etc.

In this case, the control module 170 transmits operation information of the sound wave electric module (e.g., driving start time information of the electric toothbrush, usage time information of the electric toothbrush, information on the number of times of use of the electric toothbrush, etc.) and information on power supply to the cover part 200 through the power output terminals 150 provided in the body part 100 (e.g., sterilization treatment start time information, sterilization treatment duration information, information on the number of times of sterilization treatment, etc.) to the user terminal through the communication module, such that the user may check the information.

In addition, for embodying the present invention, the control module 170 may also receive the sterilization duration information of the toothbrush head 110, which is set by the user using the user terminal, through the communication module provided in the body part 100 of the electric toothbrush, and thereby may also set the sterilization duration of the toothbrush head 110 based on the sterilization duration information received through the communication module.

As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

While the present invention has been described with reference to the preferred embodiments and modified examples, the present invention is not limited to the above-described specific embodiments and the modified examples, and it will be understood by those skilled in the related art that various modifications and variations may be made therein without departing from the scope of the present invention as defined by the appended claims, as well as these modifications and variations should not be understood separately from the technical spirit and prospect of the present invention.

### DESCRIPTION OF REFERENCE NUMERALS

100: Body part
110: Electric toothbrush head
130: Input module
150: Power output terminal
170: Control module
200: Cover part
210: Sterilization indicator
230: Open slit
250: Power input terminal
270: Sterilization lamp

What is claimed is:

1. An electric toothbrush comprising:
a body part which includes an electric toothbrush head; and
a cover part detachably coupled to the electric toothbrush head to sterilize the electric toothbrush head.

2. The electric toothbrush according to claim 1, wherein the body part includes power output terminals provided therein to supply a power to the cover part.

3. The electric toothbrush according to claim 2, wherein the cover part includes a sterilization lamp provided therein to emit light by the power supplied through the power output terminals.
